(19) United States
(12) Patent Application Publication
(10) Pub. No.: US 2007/0134354 A1
(43) Pub. Date: Jun. 14, 2007

(54) Process for producing a grape seed extract having a low content of monomeric polyphenols

(75) Inventors: Mauro Angeletti, Civitanova Marche (Macerata) (IT); Luca Sparapani, Cambrino (Macerata) (IT)

Correspondence Address: YOUNG & THOMPSON, 745 SOUTH 23RD STREET, 2ND FLOOR, ARLINGTON, VA, 22202, US

(73) Assignee: BIOSISTEMA S.R.L., Camerino (Macerata) (IT)

(21) Appl. No.: 10/575,235

(22) PCT Filed: Oct. 7, 2004
(86) PCT No.: PCT/EP04/11301

§ 371 Date: Oct. 6, 2006

(30) Foreign Application Priority Data
Oct. 8, 2003 (IT) AN2003 A 000053

(51) Int. Cl. A61K 36/87

(52) U.S. Cl. 424/766

(57) ABSTRACT

A process of grape seed extraction from Vitis vinifera grapes previously treated in any variety of manners is characterised in that skin and seeds are separated immediately after the work up to which the grapes have been subjected, so as to avoid and/or stop any fermentation due to the presence of skin of any other process liquids.

This invention refers to a process of grape seed extraction for the Vitis Vinifera species, in particular for the extraction of polyphenolic substances from grape pomace. Grape seed extraction, normally carried out on grape pomace resulting from wine processing, allows to obtain some important molecules, in particular polyphenols.

Polyphenols are helpful as integrators or additives in food production, and thus their production at high concentration and purity, and at a relatively low cost, is important. Some factors regarding the raw materials and their influence on polyphenol quality and quantity in plants have been the object of studies, as influence of different factors like stress induction (Cantos E, 2001; Rivero R M 2001), ripening grade (Lattanzio V, 2001; Gooding P S, 2001; Kennedy J A, 2000; Chevalier T, 1999; Murata M, 1997; Joy R W 4th, 1995), and cultivar type (Chang S, 2000; Sanoner P, 1999.

To date there is insufficient understanding of the influence of critical factors, such as the presence of alcohols derived from fermenting materials used for further separation of the intended species (fermentation due to the stocking of raw materials previous to their processing or, in the case of grape seeds, due to the alcoholic residues deriving from vinification).

Other unexplored factors are the correlation between quantity and quality of polyphenols from organic cultivars.

In organic cultivars, in which there is a relative absence of synthetic substances, the previously described factors of ripening grade, cultivar type, and stress from pathogenic agents sometimes occur simultaneously. Organic grape juice from specific cultivars has shown a low content of monomeric polyphenols, in particular catechin and epicatechin, the two main monomeric components of grape seeds.

The low monomer content gives grape juice from organic cultivars better features than those of other products already on the market, which are rich in monomers, such as green tea extracts while at the same time avoiding undesired side effects.

The polyphenols present in vegetal matrices can be found in monomeric form or as polimer-oligomer having low molecular weight (LWMP) and as polymers having high molecular weight (HWMP). The extraction technologies can strongly influence the quality of the final product obtained.

Polyphenol extraction, for example, from vegetal matrix is conditioned by factors like temperature, pH changes, etc. (Garcia L A, 1985; Molinelli A, 2002; Scalia S 1999). The product resulting from the extraction can change in terms of quality and quantity, according to the extraction method used (Saucier C, 2001; Wu T 2002)

Because the consumption of some species is related to some mutagenic risks (NLM (2000a) CCRIS (Chemical Carcinogenesis Research Information System), National Library of Medicine, Bethesda, Md., searched July 2000 [Record Nos. 1960, 3256, 6855, 7097, 7127]), a critical factor is to keep under control the fraction of certain monomers during all the extraction stages.

Interest and development in polyphenol extraction from grape seeds is testified to by several patents. U.S. Pat. No. 4,320,200 by Yokohama and others discloses a method to obtain anthocyanidic colorants from natural products like grapes by mixing the raw materials with a solution containing sulphite, at a temperature of at least 85° C. U.S. Pat. No. 4,320,009 by Hilton and others regards the development of a method for obtaining polyphenolic extracts from grape skin, through preparation of an aqueous extract and a further adsorbtion on a ion-exchange resin, an eluition step and if necessary a further chromatographic step on paper or on a silica thin layer gel.

This method leads to a low concentration of polyphenols. U.S. Pat. No. 4,452,822 by Shrikhande and others discloses a method involving the extraction of anthocyanines from grape pomace or other sources, extracting with sulphur bioxid followed by treatment with enzymes to eliminate solid substances present in the extract. U.S. Pat. No. 4,481,226 by Crosby and others shows the use of tannic acid to stabilise the extract. U.S. Pat. No. 4,500,556 by Langston and others deals with the treatment of grape pomace with H₂SO₃ in order to make a complex, the recovery process of which involves treating the liquid extract with a non ionic adsorbent in order to adsorb the complex.

The adsorbent bed is then treated with water to remove the materials soluble in water like sugars, organic acids and solid particles.

The complex is then eluted from the adsorbent bed with an organic acidified solvent, and then at the end the anthocyan, through eluting, is polimerised.

The solvent used in this method is preferably 100% ethanol acidified with a small quantity of mineral acid to destroy the complex, in order to free the anthocyan from HSO₃. U.S. Pat. No. 5,141,611 by Ford use a polyamidic resin with controlled porosity and surface in order to remove polyphenolic substances from an extract. Extracting most of the flavonoid substances present in grape seeds requires a technology capable of very accurate separation, especially if we need to select some groups of molecules.

Among the best patents is the that of Indena (U.S. Pat. No. 5,484,594 by Frangi et al) which enables selective polyphenol extraction, and eliminates most of the monomers with undesirable side effects.

This process makes an enriched extract of procyanidin oligomers with the use of solvents: acetone and methanol (as primary solvents), ethilacetate, methilenchloride and dichlorethane.

The main disadvantages of this process are:

the use of solvents does not allow the extraction of many helpful proantocyanidines.

the procedure needs many steps, like filtrations, incubations and is very time consuming and work intensive.

the use of carcinogenic chlorinated solvents that can cause pollution problems and difficulties in industrial hygiene

the total elimination of acetone, methanol or acetate is not possible without the formation of an azeotropic mixture.

Currently, all extraction processes start from grape pomace, containing both skin and seeds. These are passed through a de-alcoholizer which removes alcohol through all matrices.

The waste of this process is sent to a drier, which reduces humidity at a level of about 4%, and then on a separating sieve or on a seed separator that separates the skin from the seeds. This process has several disadvantages, including polyphenol extraction during the de-alcoholisation process, and elevated monomeric fraction due to the passage in the drier.

This last treatment is normally done at high temperature and for short periods of time, to minimise production times and decrease costs. Due to the low quality of the seeds utilised in these processes, the seeds are often utilised as a combustion source for driers or employed to produce seed oil, due to their high content of unsaturated oils. To date there is no practical process for selectively producing polyphenols from grape seeds and for reducing their content of monomers.

These problems are brilliantly solved by this invention, for extracting grape seeds processed in any way, characterised in that seeds and skin are separated immediately after processing, before any fermentation process can occur. A second valuable aspect of the this invention is an extract having high polyphenol content and a low content of monomeric substances obtained through the above process. In addition, the product of the present invention can be used as an additive or alimentary integrator. Finally, this invention refers the use of the seeds obtained from the extraction process waste to produce seed oil.

A detailed description of the invention follows, referring to the annexed drawings, wherein:

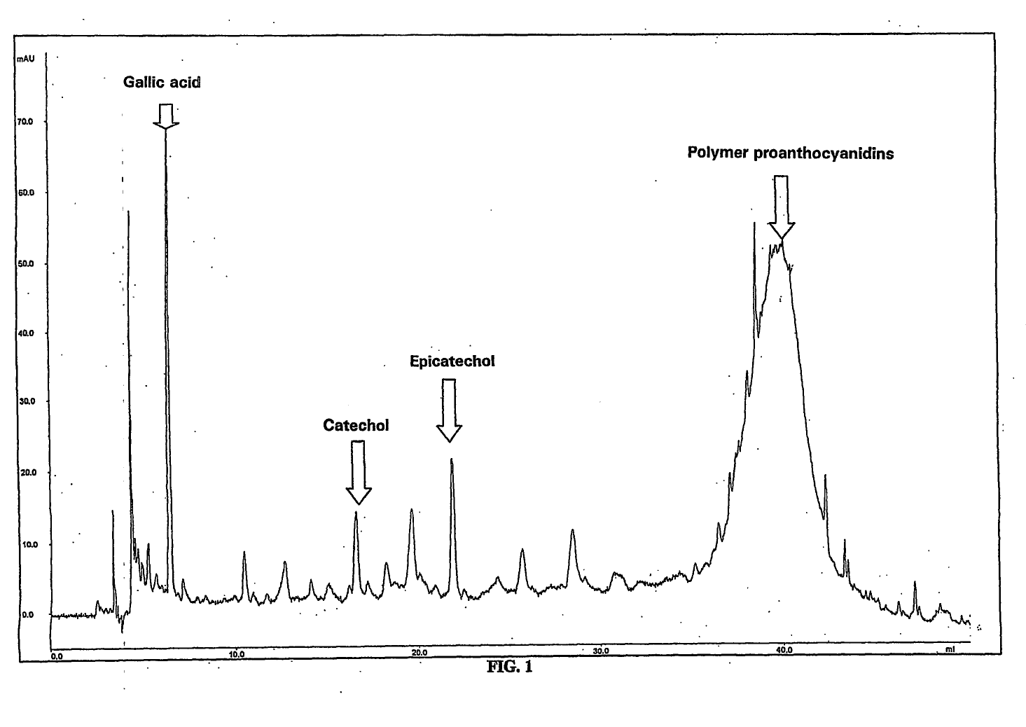

FIG. 1 is a HPLC-TV chromatogram of an extract made with the present invention; and

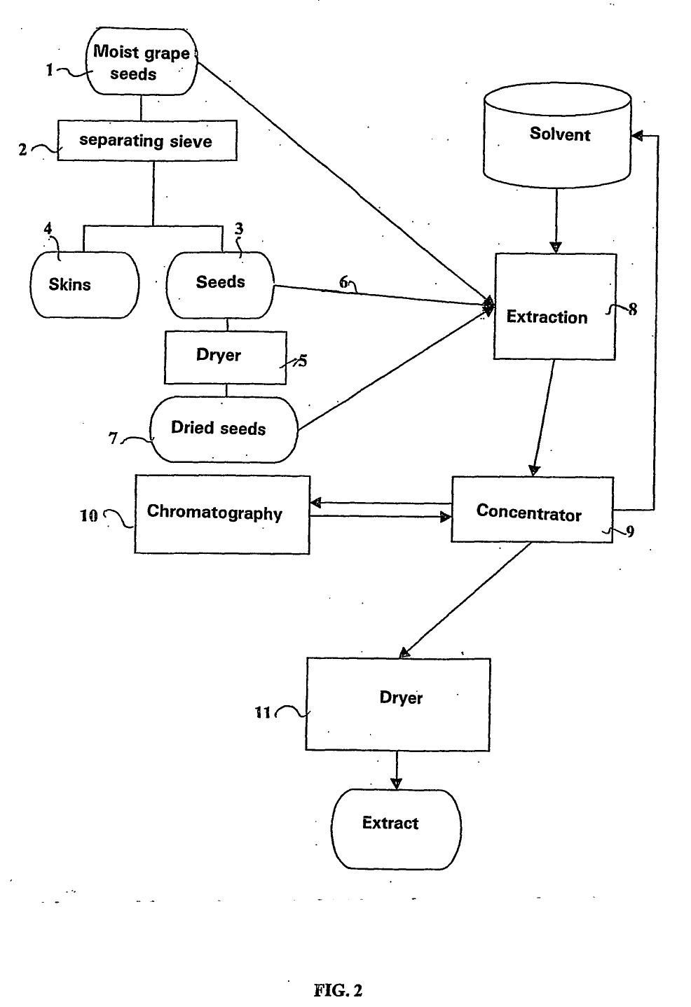

FIG. 2 is a block scheme of the extraction process according to the invention.

The proposed extraction method follows a path (illustrated in FIG. 2) different from the one currently used in modern industrial distillery, the biggest production source of seed processing. The pomace of fermented or non fermented grapes (1) resulting from the vinification process, from grape juice production or any other treatment form of grapes is separated (2) into its fundamental components (skin, seeds and rasp).

Optimal machinery for this stage can be a single or multiple stage shaking separator or a seed-separator machine.

According to our process, the separation should be taken immediately after the main processing, in order to avoid or block any fermentation that would lower the polyphenol concentration.

In this way, any de-alcoholisation processes or preventive drying can be avoided, with obvious economic advantages.

Obviously, with already separated seeds (3) this separation step is not required.

The processed skins (4) can be used for other purposes.

If necessary, the skins are passed through a de-alcoholisator. The separated seeds (3) can be processed through a drier (5).

Hot air driers are useful because they avoid the emission by combustion hoods and any contamination from hydrocarbons or other organic or inorganic sources.

The drying process should be done at a temperature between 30° C. and 120° C., until the material reaches a humidity tenor between 2% and 30%, or any other value capable of avoiding any growth of bacterial, yeast or any other microbiological agents that can lead to a fermentation process or any other form of degradation of the product.

In case of immediate extraction the drying step can be avoided (6). The seeds obtained (7) can be used for any other purpose, including the production of grape seed oil or of extract from whole or ground seeds. For the production of a low monomer content extract, any solvent capable of extracting from the vegetal matrix the interesting molecules should be used as extracting mixture; in this case, a mixture of water:ethanol (for example, 30:70) with a drug-liquor ratio of 1:10, is preferable until the solvent's saturation extractive capacity is attained.

The extraction treatment can be done in a low oxygen, tenor and with high pressures. The liquor is then separated from the exhausted seeds and the latter can be used for any other purpose, including, grape seed oil production.

The liquor is then concentrated (9) to eliminate the non aqueous phase and the product thus obtained can be purified with a chromatographic step (10), using polystirenic resins like XAD-16, XAD-4 or DIAION HP-20. If a non purified product is required, the eluate or the product from the concentrator should go through a concentration step to recover the eluent or to concentrate the product, and then a further drying (11) through spray-drying or any other process capable of making fine particles. The product with a high polyphenol content and low monomer content can be employed per se for the uses described above, and in particular, according to the present invention, such an extract can serve in the preparation of additives and alimentary integrators for food and beverages. The product can also be mixed with green tea extract, rich in monomers without any genotoxic activity.

Example: The grape pomace, obtained from press filter utilised for the production of biologic red grape juice, is separated during 72 hours with a double face separator.

The seeds thus obtained are dried on a hot air flow dryer at a temperature not superior to 60° C. for about 4 hours, until a humidity grade of 7%.

The obtained seeds are then treated with an ethanol-water mixture (70:30) utilising a 1:3 drug-solvent ratio in 4 cycles of 1 hour at a temperature of 40-50° C.

The residual seeds from the work up are removed utilising a filtre screen and the liquid phase is concentrated to eliminate ethanol.

The recovered ethanol could be reutilised for other extractions.

The aqueous phase containing the prime extract can be placed in the spray-dryer to obtain a prime product with a total polyphenol title (utilizing a Folin type analysis in terms of gallic acid equivalents) inferior to 35%. Otherwise it can be purified using a resin HP-20 Supelco, and next eluited with ethanol at 70%.

The liquid obtained is concentrated and then placed in the spray-dryer to obtain a purified product superior to 70% in terms of total polyphenols.

1) Process of grape seed extraction from Vitis vinifera grapes previously treated in any variety of manners, characterised in that skin and seeds are separated immediately after the work up to which the grapes have been subjected, so as to avoid and/or stop any fermentation due to the presence of skin of any other process liquids.

2) Process as claimed in claim 1) characterised in that said grapes come from processes not of fermentation type, ex. production of grape juice.

3) Process as claimed in claim 1) for seeds coming from organic cultivation.

4) Process as claimed in claim 1), characterised in that the separation is performed with a single or multiple step shaking separator, otherwise seedspreader machine.

5) Process as claimed in any previous claim 1), characterised in that seeds are dried after the separation.

6) Process as claimed in claim 5), characterised in that said drying step is performed in a hot air dryer.

7) Process as claimed in claim 5), characterised in that said drying step occurs at a temperature between 30 and 120° C.

8) Process as claimed in claim 5), characterised in that a humidity grade between 2 to 30% is obtained at the end of the drying step.

9) Process as claimed in any previous claim 1), characterised in that an ethanol-water mixture is used as extractor solvent.

10) Process as claimed in claim 3), characterised in that the ethanol-water mixture has a weight ratio of 30:70.

11) Process as claimed in claim 1), characterised in that a drug-liquor ratio is 1:10.

12) Process as claimed in claim 1), characterised in that an extraction step is carried out in an atmosphere with a low percentage of oxygen.

13) Process as claimed in claim 1), characterised in that an extraction step is carried out under pressure.

14) Process as claimed in claim 1), characterised in that the concentration of the liquor is carried out (9) to eliminate the non aqueous phase.

15) Process as claimed in claim 14), characterised in that purification of the product obtained is carried out using chromatography (10).

16) Process as claimed in claim 15), characterised in that a chromatographic purification step takes place onpolystyrenic resins such us XAD-16, XAD-4, DIAION HP-20.

17) Process as claimed in claim 16), characterised in that a concentration of the eluted part takes place.

18) Process as claimed in claim 1), characterised in that the concentrated eluate or any product coming from the concentrator, when an unpurified product is desired, is dried on a spray-drier or any other applicable process.

19) Grape seed extract with high polyphenol contents and low content of monomeric substances, obtained with the process according to claim 1).

20) Extract as claimed in claim 16), characterised in that it is mixed to a green tea extract.

21) Use of seeds recovered from a process as in claim 1) to produce grape seed oil.

22) Use of an extract as claimed in claim 19) as food additive or integrator.
